(19) United States
(12) Patent Application Publication
(10) Pub. No.: US 2016/0331339 A1
(43) Pub. Date: Nov. 17, 2016

(54) Systems And Methods For Early Detection And Monitoring Of Osteoarthritis

(71) Applicant: The Trustees of Columbia University in the City of New York, New York, NY (US)

(72) Inventors: Xiangdong Guo, New York, NY (US); Qi Wang, New York, NY (US); Yue Yu, New York, NY (US); Xingjian Zhang, New York, NY (US); Yizhong Hu, New York, NY (US)

(21) Appl. No.: 15/153,985

(22) Filed: May 13, 2016

Related U.S. Application Data
(60) Provisional application No. 62/238,872, filed on Oct. 8, 2015.
(60) Provisional application No. 62/162,125, filed on May 15, 2015.

(51) Int. Cl. A61B 6/00; A61B 6/03

(52) U.S. Cl. CPC A61B 6/505; A61B 6/035; A61B 6/5217

(57) ABSTRACT

The present disclosure provides systems and methods useful in early detection and assessment of osteoarthritis and other bone-related conditions.

### CROSS-REFERENCE TO RELATED APPLICATIONS

This application claims the benefit of and priority to U.S. Provisional Patent Application No. 62/238,872, filed Oct. 8, 2015, “Systems and Methods for Early Detection and Monitoring of Osteoarthritis,” and also the benefit of and priority to U.S. Provisional Application No. 62/162,125, filed May 15, 2015, “Systems And Methods For Early Detection And Monitoring Of Osteoarthritis.” The entirety of each of these applications is incorporated by reference herein for all purposes.

### GOVERNMENT RIGHTS

The subject matter disclosed herein was made with government support under grant number R01AR051376 awarded by the National Institutes of Health (NIH). The government has certain rights in the herein disclosed subject matter.

### TECHNICAL FIELD

The present disclosure relates to the field of osteoarthritis assessment.

### BACKGROUND

Osteoarthritis (or OA) is a debilitating musculoskeletal disease that affects approximately 27 million aging adults in the United States and an estimated 9.6% of men and 18.0% of women over the age of 60 worldwide. In younger patients, traumatic injuries, such as anterior cruciate ligament (ACL) tears, often lead to the early onset of OA.

Currently, clinical outcomes for osteoarthritis OA are discouraging and clinical diagnosis and research on the pathogenesis of OA and development of effective therapeutic options are severely hindered because current diagnostic and monitoring techniques are unreliable and insensitive, only providing a macroscopic view of the joint. For these reasons alone, there is a great unmet need for a reliable and sensitive tool to assess OA pathological changes.

Despite its prevalence, the pathogenesis of OA remains largely unknown. Thus, it is of high clinical impact to elucidate the mechanisms underlying the development and progression of OA.

Further, existing therapeutic options for OA are largely palliative and cannot prevent disease progression, necessitating surgery as the only long-term treatment most individuals. Conventionally considered a disease in the cartilage, OA has been assessed by physical examination and plain radiographs. These methods are insensitive and inconsistent, making it difficult to confirm diagnosis. As a result, OA is not typically confirmed until its later stage when tissue damage becomes irreversible, thus missing the ideal time window for clinical intervention.

In addition, the cycle of care for OA is complex, involving multiple healthcare facilities and professionals including primary care physicians, physical therapists, orthopedic specialists, and surgeons. Patients are regularly transferred back and forth between healthcare professionals, incurring additional monetary and time cost. After patient evaluation, non-pharmacological treatment and pharmacological treatment are prescribed sequentially. Available pharmacological treatment options are largely palliative with the use of opioids, non-steroidal anti-inflammatory drugs and steroid injections. As disease progresses, joint replacement surgeries and prostheses—costing on average about $15K per joint and adding about $15B to the national cost of OA—become the only long-term aid in some individuals. Accordingly, there is a long-felt need in the art for methods and systems that allow for timely detection, assessment, and treatment of OA.

### SUMMARY

In meeting these long-felt needs, the present disclosure first provides methods, the methods comprising determining, for a bone sample, one or more of (1) a volume fraction of trabecular bone plates to total bone volume, of trabecular bone rods to total bone volume, or both, (2) a cross-sectional dimension of trabecular bone plate, of trabecular bone rod, or both, (3) a number density of trabecular bone plates, of trabecular bone rods, or both, (4) an elastic modulus, and (5) one or more ratios that include any one or more of the foregoing; (b) comparing (1), (2), (3), (4), (5) or any combination thereof to one or more comparative quantities; and (c) estimating a degree of osteoarthritis, osteoporosis, or both, of the bone sample.

Other disclosed methods comprise providing a CT scanning device operatively connected to an osteoarthritis estimation component that comprises a processor; operating the CT scanning device so as to obtain bone structural information from a bone sample; outputting the bone structural information to the osteoarthritis estimation component; and executing instructions on the processor of the intracranial pressure estimation component, the instructions including (a) receiving the bone structural information, and (b) generating from that information one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, and estimating a degree of osteoarthritis, osteoporosis, or both, in the bone sample from one or more of the foregoing quantities (1)-(5).

The present disclosure also provides methods of treating osteoarthritis, comprising: administering a therapeutically effective dose of an osteoarthritis treatment to a patient who is asymptomatic for osteoarthritis and exhibits one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, wherein the foregoing characteristic of the patient differs from one or more comparative quantities.

Further provided are diagnostic systems, the systems comprising a toroid tomography scanner, the toroid having a central axis and an inner diameter about the central axis, the inner diameter of the toroid being so dimensioned as to permit entry of a human limb such that a joint of the limb is disposed within the toroid, the toroid comprising an x-ray source and an x-ray detector, the source and detector configured to be in x-ray communication with one another during operation, the system being configured such that one or more of the x-ray source, the x-ray detector, and the toroid are rotatable about the limb disposed within the toroid.

Other disclosed systems comprise a processor configured to determine, structural information of a bone sample, one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

The summary, as well as the following detailed description, is further understood when read in conjunction with the appended drawings. For the purpose of illustrating the disclosed subject matter, there are shown in the drawings exemplary embodiments of the disclosed subject matter; however, the disclosed subject matter is not limited to the specific methods, compositions, and devices disclosed. In addition, the drawings are not necessarily drawn to scale. In the drawings:

FIG. 1 provides an exemplary individual trabecula segmentation (ITS) analysis and plate/rod distribution of an exemplary trabecular bone microstructure;

FIG. 2 provides tibial subchondral and femoral neck plate and rod distributions by orientation (different scales apply to each y-axis);

FIG. 3 provides macroscopic and radiographic view of tibial plateau from non-OA donors and severe OA patients;

FIGS. 4 and 5 provide histological images and finite element analysis of lateral and medial sides (respectively) of tibial plateau from non-OA and OA-affected knees of FIG. 3;

FIG. 6 provides ITS analysis of lateral and medial side subchondral bone in non-OA and OA-affected knees from FIGS. 4 and 5;

FIG. 7 provides radiographic images, histology, and ITS analysis of tibial plateaus from an illustrative animal (guinea pig) study;

FIGS. 8 and 9 provide pTb.S (plate trabecular spacing) and rTb.l (mean trabecular rod length) results for control and OA animals at 1, 2, and 3 months;

FIGS. 10 and 11 provide r-r Junc.D (rod-rod junction density) and p-r Junc.D (plate-rod junction density) results for control and OA animals at 1, 2, and 3 months;

FIGS. 12 and 13 provide p-p Junc.D (plate-plate junction density) and p-r ratio results for control and OA animals at 1, 2, and 3 months;

FIG. 14 provides a proposed, non-limiting schematic for OA pathogenesis;

FIG. 15 provides a depiction of an exemplary device according to the present disclosure;

FIG. 16 provides a further depiction of an exemplary device according to the present disclosure;

FIG. 17 provides a depiction of an exemplary scanner component of a device according to the present disclosure; and

FIG. 18 provides an alternative depiction of the components of a scanner component; the dashed circle shows the trajectory of the x-ray source and detector rotating so as to encircle a sample, with the rotating being in the direction of the counterclockwise arrows.

Further background on the nomenclature used herein may be found in Liu et al., J. of Bone and Mineral Res. 2008:23:223-235; and Wang et al., Osteoarthritis and Cartilage 2013:21:574-581. The terminology may also be summarized as follows:

| Parameters | | | |
|---|---|---|---|
| Abbreviations | Full names | Definition | Unit |
| pBV/TV | Plate bone volume | The total volume of trabecular plates divided | % |
| | fraction | by the bulk volume | |
| rBV/TV | Rod bone volume | The total volume of trabecular rods divided | % |
| | fraction | by the bulk volume | |
| aBV/TV | Axial bone volume | The total volume of trabeculae aligned with | % |
| | fraction | the longitudinal axis divided by the bulk | |
| | | volume | |
| pBV/BV | Plate tissue fraction | The total volume of trabecular plates divided | % |
| | | by the total bone volume | |
| rBV/BV | Rod tissue fraction | The total volume of trabecular rods divided | % |
| | | by the total bone volume | |
| pTb.N | Trabecular plate | The cubic root of the total number of | 1/mm |
| | number density | trabecular plates divided by the bulk volume | |
| rTb.N | Trabecular rod | The cubic root of the total number of | 1/mm |
| | number density | trabecular rods divided by the bulk volume | |
| pTb.Th | Mean trabecular | The average thickness of trabecular plates | μm |
| | plate thickness | | |
| rTb.Th | Mean trabecular rod | The average thickness of trabecular rods | μm |
| | thickness | | |
| pTb.S | Mean trabecular | The average surface area of trabecular plates | 10³ |
| | plate surface area | | μm² |
| rTb.1 | Mean trabecular rod | The aver-age length of trabecular rods | μm |
| | length | | |
| R-R Junc.D | Rod-rod junction | The total number of junctions between rod | 1/mm |
| | density | and rod divided by the bulk volume | 3 |
| P-R Junc.D | Plate-rod junction | The total number of junctions between plate | 1/mm |
| | density | and rod divided by the bulk volume | 3 |
| P-P Junc.D | Plate-plate junction | The total number of junctions between plate | 1/mm |
| | density | and plate divided by the bulk volume | 3 |
| P-R ratio | Plate-to-rod ratio | The total volume of trabecular plates divided | — |
| | | by the total volume of trabecular rods | |

### DETAILED DESCRIPTION OF ILLUSTRATIVE EMBODIMENTS

The present disclosed subject matter may be understood more readily by reference to the following detailed description taken in connection with the accompanying figures and examples, which form a part of this disclosure. It is to be understood that this disclosed subject matter is not limited to the specific devices, methods, applications, conditions or parameters described and/or shown herein, and that the terminology used herein is for the purpose of describing particular embodiments by way of example only and is not intended to be limiting of the claimed subject matter. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the disclosed subject matter which are, for clarity, described herein in the context of separate embodiments, may also be provided in combination in a single embodiment. Conversely, various features of the disclosed subject matter that are, for brevity, described in the context of a single embodiment, may also be provided separately or in any subcombination. Further, reference to values stated in ranges include each and every value within that range.

Human trabecular bone comprises individual trabecular plates and rods, and an advanced three-dimensional (3D) automated, imaging analysis technique, individual trabecula segmentation (ITS), completely decomposes the 3D trabecular bone microstructure into a collection of individual trabecular plates and rods. ITS-based morphologic analysis based on tomography images of human trabecular bone provides additional plate and rod microstructural measurements beyond traditional morphological parameters.

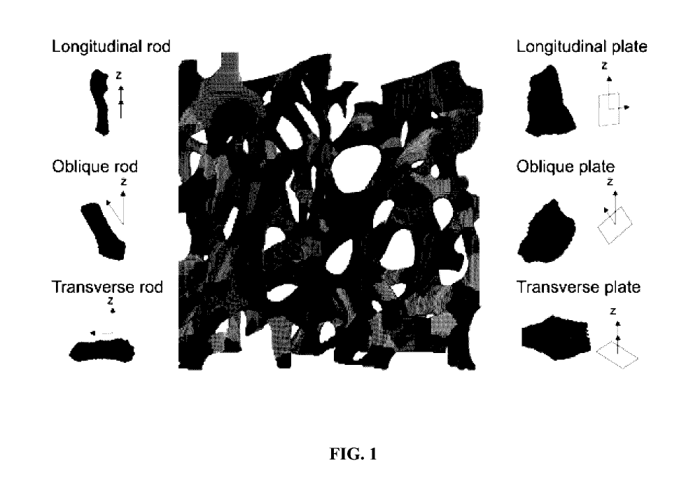

As described herein, ITS-based microstructural analyses have demonstrated that—unexpectedly—bone plate volume fraction (pBV/TV) and axial bone volume fraction (aBV/TV), rather than bone rod volume fraction (rBV/TV), determine trabecular bone mechanical properties. Trabecular bone is shown in FIG. 1, which figure depicts trabecular rod and bone plate, along with trabecular orientation. (It should be understood that “trabecular rod bone” may be interchanged with “trabecular bone rod” and that “trabecular bone plate” may be interchanged with “trabecular plate bone.”)

It has been demonstrated that trabecular plates and rods influence both elastic modulus and yield strength of human trabecular bone. The basic science and clinical applications of ITS imaging analysis in osteoporosis indicate the major loss of trabecular plates in osteoporotic bone loss.

This disclosure provides a novel, nonobvious application of ITS plate and rod analysis technique in subchondral bone samples in human osteoarthritis (OA). Subchondral bone sclerosis and stiffening are well known in OA. However, the recent advances suggest that increased and abnormal bone remodeling, particular bone resorption, precedes the development of OA.

Using ITS analysis of subchondral trabecular bone samples from human OA knees, it was discovered that the early sign of abnormal subchondral trabecular bone remodeling in OA begins with a dramatic loss of trabecular rods before any significant changes in BV/TV, i.e., even while a patient is asymptomatic. These changes lead to adaptive bone responses by dramatically thickening the remaining trabecular plates and rods, resulting in subchondral bone sclerosis and stiffening. The resulting subchondral trabecular bone can be characterized as sparsely spaced thick trabecular plates in the axial direction, providing an uneven and ridged subchondral bone bed for overlaying cartilage. These trabecular plate and rod changes in OA provide morphological and biomechanical insight into abnormal subchondral bone remodeling in the etiology of osteoarthritis.

FIG. 1 depicts an exemplary ITS analysis and plate/rod distribution for an exemplary bone sample. The figure also provides the orientation (longitudinal, oblique, and transverse) of the rods and plates in the sample.

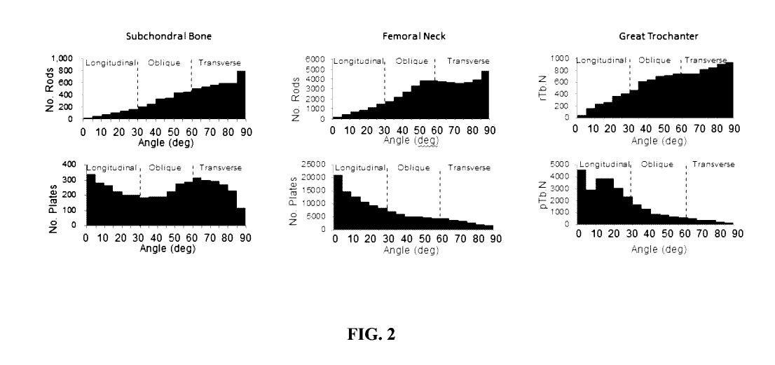

FIG. 2 provides exemplary plate and rod distributions (by angle) for subchondral, femoral neck, and great trochanter bone. As seen, the distribution of rods and plates may vary depending on the bone sample. ITS analysis revealed certain unappreciated trabecular bone characteristics of the subchondral bone of the knee compared with the femoral neck, another load bearing site: trabecular plates orient in both longitudinal and transverse directions in subchondral bone. However, while trabecular rods orient predominately in the transverse direction in both anatomic locations, the proportion of plates and rods differ: subchondral bone consists mostly of trabecular rods, while femoral neck mostly consists of trabecular plates. These observations are novel and consistent with the role of subchondral bone in transmitting joint loads from soft cartilage to the supporting skeleton.

Application of applied ITS to tomography images of human knees revealed that subchondral trabecular bone in this location is predominantly composed of compliant trabecular rods. In addition, not only were detected significantly fewer subchondral trabecular rods but also significantly more trabecular plates beneath damaged articular cartilage in human OA knees. Interestingly, also detected were significantly fewer rods beneath the intact articular cartilage of the same OA-affected knees. This suggests that early rod loss, which cannot be observed by conventional techniques, may precede cartilage degradation in early OA. The divergent changes between trabecular plates and rods suggest that the pathogenesis of OA is unique and can be explored only by ITS.

Similar rod loss that antedated cartilage degradation was also observed in a spontaneous OA guinea pig model, described elsewhere herein. These analyses suggest a mechanism for OA pathogenesis that is driven by bone biomechanics: loss of compliant trabecular rods destabilizes subchondral bone, leading to chronic increases in stiffer trabecular plates, and initiates cartilage degradation that ultimately progresses to OA. This mechanism is supported both by the fact that patients with ACL injuries exhibit early subchondral bone loss and that ACL reconstruction does not prevent OA suggesting that the early changes observed after ACL injuries persist, ultimately leading to chronic OA. Without being bound to any particular theory, application of ITS to high-resolution peripheral quantitative CT (HR-pQCT) can identify preferential loss of trabecular rods in subchondral bone of patients with OA. One may also detect rod loss in patients after acute ACL tears, establishing rod loss as an early and persistent biomarker of OA.

In addition to age and obesity, ACL tears are one of the strongest risk factors for OA. ACL tears are common knee injuries in young, active people. More than 50% of patients with ACL tears develop OA within 10-20 years. Although surgical re-pair of ACL tears restores most knee joint functionality and patients return to normal daily activities, surgery has minimal impact on the long-term risk of OA. In addition to pathological changes in articular cartilage immediately following ACL injuries, X-ray or CT studies have detected subchondral bone loss as early as a few weeks post-injury, which may plateau 6-8 months after an ACL injury.

It is generally accepted that this early bone loss in subchondral bone is followed by an eventual increase in bone density, and these observations are consistent with results of many animal studies. The bone loss may return to baseline after one year or remain below the values of the unaffected contralateral limb. How this early bone loss and recovery gradually lead to significant sclerosis of subchondral bone, however, remains a mystery.

One may hypothesize that there is a significant loss of trabecular rods in the acute phase of ACL injuries, followed by subsequent thickening of trabecular plates and rods. Accordingly, ACL-injured patients are a population well-suited to the present technology.

In meeting these long-felt needs, the present disclosure first provides methods. The methods may comprise (a) determining, for a bone sample, one or more of (1) a volume fraction of trabecular bone plate to total bone volume, of trabecular bone rod to total bone volume, or both, (2) a cross-sectional dimension of trabecular bone plate, of trabecular bone rod, or both, (3) a number density of trabecular bone plate, of trabecular bone rod, or both, (4) an elastic modulus, (5) one or more ratios that include any one of the foregoing (e.g., a volume ratio), or any combination of the foregoing.

As some examples, one may determine the volume fraction of trabecular bone rod (rBV), the volume fraction of trabecular bone plate (pBV), the ratio (e.g., P-R, or pBV/rBV) of trabecular bone rod to trabecular bone plate, the ratio of trabecular bone rod to total bone volume (rBV/TV), the ratio of trabecular bone plate to total bone volume (pBV/TV), or any combination thereof.

The methods may suitably comprise comparing (1), (2), (3), (4), (5) or any combination thereof to one or more comparative quantities, e.g., one or more comparative quantities of a control sample that are considered threshold values. For example, one might compare the rBV/TV value of a patient sample to the rBV/TV value of a control sample or the rBV/TV value for a sample taken from that patient at an earlier point in time or even to an average value for a population.

The methods also include estimating a degree of osteoarthritis, osteoporosis, or both, in the bone sample. This may be accomplished by considering the rBV/TV value of the sample; an increase in rBV/TV may suggest the onset (or further progress) of osteoarthritis in the subject.

The determining is suitably performed on a CT scanning device. The CT scan may have a resolution that allows resolution of trabecular rod and trabecular bone. The identification and assessment of trabecular bone may be accomplished in a number of ways that are known to those of ordinary skill in the art. As one example, one may use an ITS technique.

A CT scan may have a (voxel) resolution of between about 200 micrometers and about 20 micrometers, or even a resolution of between about 150 and 80 micrometers. Resolutions in the range of 50 to about 70 micrometers are considered suitable. Some exemplary peripheral quantitative CT machines may have a voxel resolution between about 200 to 1000 micrometers; a more preferable resolution is from about 55 micrometers to about 200 micrometers. Resolutions in the range of 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or even 31 micrometers are considered especially suitable. As described elsewhere herein, determining may be effected by providing x-rays of two or more different energies (or frequencies, or other characteristic) to the bone sample.

The methods may include determining one or more of the quantities listed herein (e.g., rBV, pBV, TV) for two or more locations within the bone sample. The methods may also include determining one or more of these quantities for the same sample at two or more points in time.

As described above, the methods may include determining a ratio of the volume fraction of trabecular bone rod to total bone volume (rBV/TV). The methods may also include determining a ratio of trabecular bone rod volume to trabecular bone plate volume (rBV/pBV). The methods may also include determining a ratio of trabecular bone plate volume to total bone volume (pBV/TV).

The disclosed methods may also include selecting a treatment regimen based at least in part on the determination described above. For example, if a practitioner identifies an increase in rBV/TV, the practitioner may prescribe a medication, an exercise regimen, or both, for the patient having the increased rBV/TV. As another example, if a practitioner identifies an increase in rBV/TV, the practitioner may also prescribe a change in treatment from a treatment method that the patient is already using.

Additionally provided methods include determining, for a bone sample, one or more of (1) a volume ratio of trabecular bone rod to trabecular bone plate, (2) a ratio of trabecular bone rod volume to total bone volume; comparing (1), (2), or both to one or more comparative quantities of a control sample; and estimating a degree of osteoarthritis, osteoporosis, or both, in the bone sample. The methods may include determining (1), (2), or both for two or more locations within the bone sample. One may also determine (1), (2), or both for a sample at two different points in time.

As described elsewhere herein, determining the structure of the bone sample may be based on a CT scan of the bone sample. Other scan/image modalities (e.g., MRIs) are also suitable, and the present methods are not limited to practice on CT scans. The present methods may be practiced on any scan/image that allows for assessment of trabecular rod, trabecular plate, or even both such types of bone. The methods may include collecting the image from which the user assesses trabecular rod, trabecular plate, or even both such types of bone.

Other methods provided herein comprise administering an osteoarthritis treatment to a patient; determining, for a first bone sample, one or more of (1) a volume fraction of trabecular bone plate to total bone volume, of trabecular bone rod to total bone volume, or both, (2) a cross-sectional dimension of trabecular bone plate, of trabecular bone rod, or both, (3) a number density of trabecular bone plate, of trabecular bone rod, or both, (4) an elastic modulus, (5) one or more ratios that include any one of the foregoing, or any combination of the foregoing; and comparing (1), (2), (3), (4), (5) or any combination thereof to one or more comparative quantities of a bone sample that differs from the first bone sample in source, time of collection, or location. The user may further assess a degree of effectiveness of the treatment.

The treatment may act to promote bone rod regrowth, to reduce or eliminate the loss of rods, to reduce or retard plate growth, or otherwise promote bone remodeling. The treatment may also act to retard bone modeling so as to slow or even stop the changes in bone structure that may occur with OA. Illustrative treatments include, e.g., NSAIDS, antiresorptive treatments, and the like. Exemplary antiresorptive treatments include bisphosphonates, hormone replacement therapy, selective estrogen-receptor modulators (SERMs), calcitonin, and denosumab. Treatment may (1) reduce inflammation induced bone loss and (2) reduce pain so that patient may continue walking to reduce unloading induced bone loss.

The present disclosure provides still other methods, these methods suitably including determining, for a bone sample, one or more of (1) a volume ratio of trabecular bone rod to trabecular bone plate, (2) a ratio of trabecular bone rod volume to total bone volume; and assessing a degree of osteoarthritis, osteoporosis, or both, in the bone sample.

Other disclosed methods include determining, for a bone sample, one or more of (1) a volume fraction of trabecular bone plate to total bone volume, of trabecular bone rod to total bone volume, or both, (2) a cross-sectional dimension of trabecular bone plate, of trabecular bone rod, or both, (3) a number density of trabecular bone plate, of trabecular bone rod, or both, (4) an elastic modulus, (5) one or more ratios that include any one of the foregoing, or any combination of the foregoing; and assessing a degree of osteoarthritis, osteoporosis, or both, in the bone sample.

Other methods provided herein may comprise providing a CT scanning device operatively connected to an osteoarthritis estimation component that comprises a processor and operating the CT scanning device so as to obtain bone structural information from a bone sample. Suitable CT scanning devices are described elsewhere herein, and can comprise micro-CT and HR-PQCT devices.

The methods also suitably include outputting the bone structural information to the osteoarthritis estimation component and executing instructions on the processor of the intracranial pressure estimation component.

The instructions may include receiving the bone structural information, generating from that information one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, and estimating a degree of osteoarthritis, osteoporosis, or both, in the bone sample, e.g., from one or more of the foregoing quantities (1)-(5).

The estimation of the osteoarthritis, osteoporosis, or both, may be performed by comparing one or more of the foregoing quantities to a comparative quantity. Such a comparative quantity may be a quantity of a control sample or some threshold value. The threshold value may be fixed or may be variable, depending on the user and the patient. For example, threshold values may be adjusted based on the age, gender, activity level, or other characteristic of the patient.

Also provided are methods of treating (pre-emptively, in some instances) osteoarthritis. These methods comprise administering a therapeutically effective dose of an osteoarthritis treatment to a patient who is asymptomatic for osteoarthritis and exhibits one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, that are greater than one or more comparative quantities, e.g., a control sample.

By “asymptomatic” is meant an individual who is experiencing little to no OA symptoms. In some instances, an “asymptomatic” individual may have an OARSI score of 2 or less. These methods allow a user to take advantage of one of the key insights of the present disclosure, namely that bone that resides beneath intact cartilage may nonetheless begin experience changes in trabecular rod and plate structures. Put another way, an individual may have the structural changes associated with OA while still remaining asymptomatic. After an asymptomatic individual is diagnosed with early (or other) stage of OA, their treating physician can provide early treatment on that individual to slow or even prevent further development of OA in that individual.

The present disclosure also provides treatment methods. These methods comprise administering a therapeutically effective dose of an osteoarthritis treatment to a patient who is asymptomatic for osteoarthritis and exhibits one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, that are greater than one or more comparative quantities of a control sample.

Other methods according to the present disclosure comprise methods of detecting osteoarthritis in a patient, said method comprising: detecting whether osteoarthritis is present in a bone sample of a subject by irradiating the bone sample and determining a value of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing. The detecting may further comprise determining whether the estimated quantity differs from a comparative value, e.g., a threshold value or the value of a control sample. A patient sample may comprise one or more tracers (e.g., a dye, a contrast agent, an antibody) that enhance the visibility of the sample during scanning. The detection of osteoarthritis may be accomplished by detecting, e.g., via CT scan or other modality, the presence of the tracer or binding or other interaction between a tracer and the patient sample. One may introduce a tracer to a subject such that the subject's tissue comprises the tracer and then detect the presence of the tracer by detecting changes in the tissue that are indicative of the tracer's presence, e.g., with a CT scan.

Further provided are methods of osteoarthritis treatment, the methods comprising administering, to a patient exhibiting osteoarthritis, an effective amount of a composition that (a) reduces loss of trabecular rods, (b) reduces thickening of trabecular plates, (c) reduces thickening of trabecular rods, or any combination thereof. The presence of OA may be determined by the methods described elsewhere herein, e.g., via determining a value of rBV/TV and comparing that sample value to a comparative value. It should be understood that the disclosed methods may be applied to patients who do not experience OA symptoms, as the structural changes associated with OA may precede the onset of physical symptoms.

Devices

High-resolution peripheral quantitative computed tomography (HR-pQCT) allows for the quantification of trabecular and cortical structure in vivo, with the capability of generating images at multiple resolutions. HR-pQCT acquires images based on the same principles as traditional QCT but can achieve a much higher resolution with the trade-off of a smaller field of view. This in turn permits only the scanning of peripheral sites such as the radius and tibia, which problem is addressed by the presently disclosed technology. Radiation exposure during an HR-pQCT scan is several orders of magnitude lower than whole body CT at an approximate 3 μSv effective patient dose per scan. HR-pQCT (and its related morphological and micro finite element (μFE) capabilities) has not been previously used to study knee OA. Because of their high levels of radiation exposure, previous scanning techniques (such as micro-CT) are limited to only peripheral sites such as the tibia and radius.

In another aspect, the present disclosure provides diagnostic systems. The systems suitably comprise a processor configured to determine, structural information of a bone sample, one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing.

The processor may be configured to compare (1), (2), (3), (4), (5), or any combination thereof to one or more comparative quantities.

The processor may also be configured to estimate a degree of osteoarthritis, osteoporosis, or both, in the bone sample. A comparative quantity may be, e.g., a value from a control sample or some other threshold value, e.g., an average value for a cohort (e.g., by age, by gender, by activity level) of the patient under analysis.

A system may also comprise an imaging device capable of providing structural information related to a subject's bone for the processor. The imaging device may be, e.g., an x-ray machine, a CT scanner (including high-resolution peripheral quantitative CT scanning devices), an MRI device, and the like. (Suitable CT scanning devices are described elsewhere herein.) It should be understood that the structural information may be saved in an electronic format and then provided to the processor. In some embodiments, the CT scanning device may be operated so as to provide x-rays of two or more different energies to the bone sample.

The present disclosure also provides computer-assisted tomography systems. The systems suitably comprise a toroid tomography scanner, the toroid having a central axis and an inner diameter about the central axis, the inner diameter of the toroid being so dimensioned as to permit entry of a human limb such that a joint of the limb is disposed within the toroid.

The inner diameter of the toroid may be, e.g., 200 mm to about 400 mm, or from about 250 to about 350 mm, or even about 300 mm.

It should be understood that a toroid may be donut-shaped, but need not be a complete circle. As one example, a toroid may be C-shaped. A toroid also need not be perfectly circular.

The toroid may comprise an x-ray source and an x-ray detector. The source and detector are suitably configured to be in x-ray communication with one another during operation.

A system may also be configured such that one or more of the x-ray source, the x-ray detector, and the toroid are rotatable about the limb disposed within the toroid. As one example, the toroid may be stationary and circular, and the x-ray source and x-ray detector may rotate within the toroid so as to give rise to a 360-degree cross-section view of the subject's limb. Alternatively, the toroid itself may rotate.

A system—including the toroid—may be configured to operate as a high resolution peripheral quantitative computed tomography system. The system may be configured to output at a resolution of from about 100 to about 40 microns per voxel, e.g., from about 90 to about 50, from about 80 to about 60, or even about 70 microns per voxel, e.g., from about 60 to about 80 microns per voxel. A system may be configured to enable adjustable voxel resolution.

The devices may also be configured to perform a so-called multiple (e.g., dual) energy scan. In this embodiment, a device according to the present disclosure may scan a sample at two or more different energy levels. These scans may be done simultaneously or sequentially. In one embodiment, this may be performed by rotating an x-ray source around a sample through a first angle (e.g., from 0 degrees to 180 degrees), during which rotation the x-ray source provides x-rays to the sample (and to an x-ray detector) at a first energy level, e.g., from about 40 to about 70 kV. An x-ray source (which may be the same as the first x-ray source or different from the first x-ray source) may then rotate through a second angle (e.g., from 181 to 360 degrees) about the sample; during this rotation, the x-ray source provides x-rays to the sample at a second energy level that differs from the first.

As one example, the first energy level may be 60 kV, and the second energy level may be 55 kV. It is preferable, though not required, that the combination of the scans sum to a full rotation (i.e., 360 degrees) around the sample of interest. In some embodiments, a system may have a peak KV of from about 60 to about 69 kV.

As another example, the first energy level may be supplied by an x-ray source operating at 60 kV. The second energy level may also be supplied by the same (or a different) x-ray source operating at 60 kV, except the second energy level is filtered by an aluminum, copper, or other filter. In this way, a user may use the same x-ray source for both scans and use a filter for one of the two scans such that the filter changes the intensity of x-rays that are delivered to the sample.

The x-rays from each of the two (or more) scans may be measured by the same x-ray detector or by different x-ray detectors. By using a multiple-energy approach, a user may visualize different materials (bone, cartilage, etc.) in a sample, as different materials absorb x-rays at different energy levels differently. It should be understood that the energy levels of scans in a multiple-energy approach may be described in terms of kV received by the sample, but they scans may also be described in terms of mAs received by the sample.

The toroid may be mounted on a portable chassis. In some embodiments, the system is configured such that it may be transported (e.g., wheeled) between rooms or other locations at a facility, such as a hospital or clinic. A chassis may comprise one or more features configured to engage with a complementary feature on a floor so as to stabilize the system. Such features include, e.g., pegs, buckles, and the like. A chassis may also comprise one or more features configured to maintain the chassis in position, e.g., bumpers, pads, wheel stops, and the like. A system may have an overall cross section of 2 meters or less, e.g., 1.9, 1.8, 1.7, 1.6, 1.5, 1.4, 1.3, 1.2, 1.1, 1.0, 0.9, 0.8, 0.7, 0.6, or even about 0.5 meters or less. A system may be cube-shaped in configuration or may be otherwise shaped so as to facilitate transportation. A system may be sized so as to fit through a doorway.

A toroid may have a thickness in the range of from, e.g., about 200 to about 400 mm. In some embodiments, the toroid may have a thickness in the range of from about 250 to about 350 mm. The thickness of the toroid (as well as a dimension of the x-ray detector, x-ray source, or of any of the foregoing) may be dimensioned such that a scan has a width sufficient to span the corresponding dimension of the joint being studied.

A system may also comprise a processor, which processor may be operatively connected to the toroid. The processor may be configured to (a) determine, based on structural information of a bone of the joint of a subject, one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing.

The processor may be configured to compare (1), (2), (3), (4), (5), or any combination thereof to one or more comparative quantities. The processor may be further configured to estimate a degree of osteoarthritis, osteoporosis, or both, in the bone sample.

A user may also employ a cast, brace, or other stabilizer to hold the subject's joint in place. Such a device may be secured via buckles, Velcro™, or other fasteners. The device may be x-ray transparent, but may also include x-ray markers to assist in positioning the subject's joint.

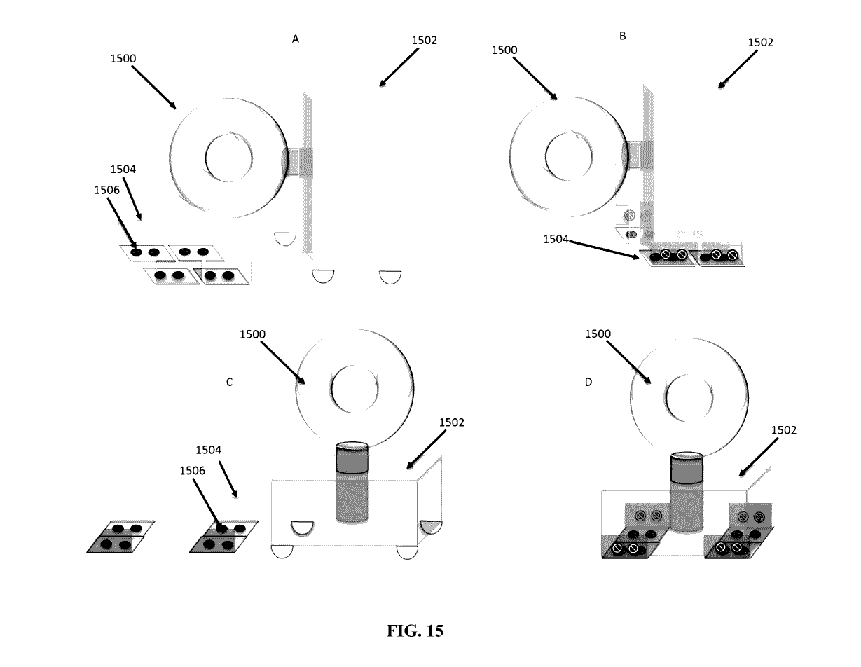

FIG. 15 depicts an exemplary system. As shown in FIG. 15A, a system may comprise toroid 1500, which toroid is connected and supported by chassis 1502. As shown in this embodiment, the toroid may extend sideways from the chassis. The chassis may be moved and then mated to bracket 1504, which bracket is secured via screws 1506. FIG. 15B depicts the system in a stabilized position, in which chassis 1502 and toroid 1500 are secured to bracket 1504.

FIG. 15C provides an alternative embodiment in which toroid 1500 extends upwards from chassis 1502. The chassis may be moved and then mated to bracket 1504, which bracket is secured via screws 1506. FIG. 15D depicts the system in a stabilized position, in which chassis 1502 and toroid 1500 are secured to bracket 1504.

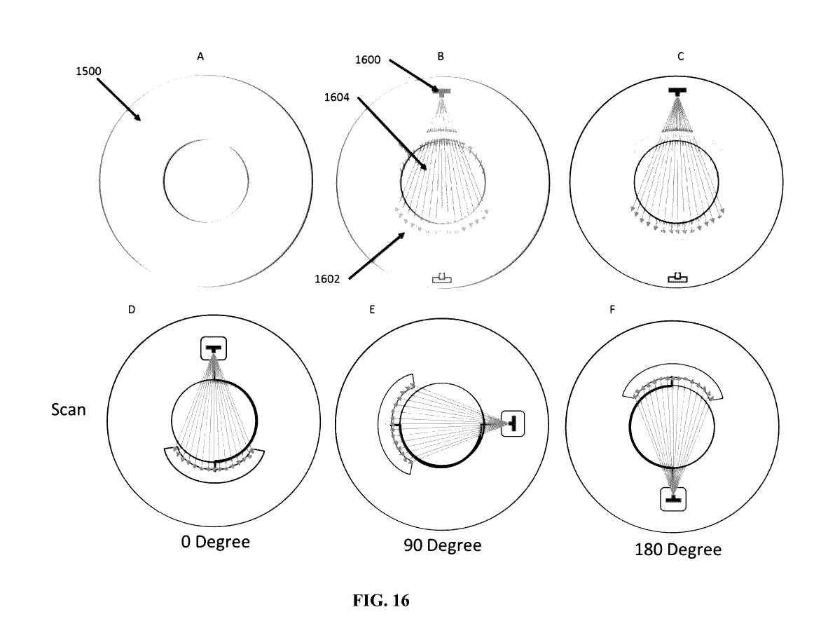

FIG. 16 provides a depiction of an exemplary operation of the disclosed systems. FIG. 16A provides an exterior view of a toroid 1500. FIG. 16B provides a component view of the interior of the toroid, showing x-ray source 1600 and x-ray detector 1602 opposite from one another, with x-rays 1604 being emitted from the source and being collected by the detector. FIG. 16C shows the direction of rotation (clockwise) of the x-ray source and x-ray detector. FIG. 16D shows the x-ray source and detector opposite from another at a position 0 degrees from the start. FIG. 16E shows the x-ray source and detector opposite from another at a position 90 degrees from the starting position, and FIG. 16F shows the x-ray source and detector opposite from another at a position 180 degrees from the starting position.

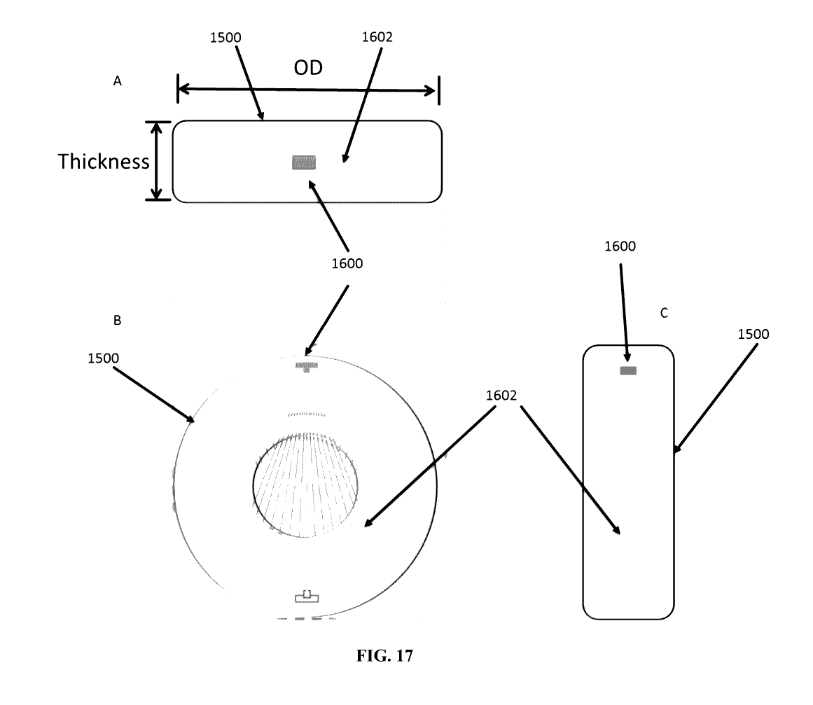

FIG. 17 provides an alternative view of the toroid 1500. As shown in FIG. 17A (a top-down cross-section view), the toroid 1500 may have disposed within an x-ray source 1600 and an x-ray detector 1602. As shown in FIG. 17B, the x-ray source 1600 may begin operation at the top of the toroid 1500 with the x-ray detector 1602 at the bottom. The source and detector may revolve during operation such that the x-rays (shown with arrows within toroid 1500) irradiate some or all of the circumference of a sample. FIG. 17C provides an alternative side cross-section view showing toroid 1500 and x-ray source 1600 and x-ray detector 1602 disposed within toroid 1500.

The present disclosure also provides systems. Systems according to the disclosure suitably include a processor configured to determine, for a bone sample, one or more of (1) a volume fraction of trabecular bone plate to total bone volume, of trabecular bone rod to total bone volume, or both, (2) a cross-sectional dimension of trabecular bone plate, of trabecular bone rod, or both, (3) a number density of trabecular bone plate, of trabecular bone rod, or both, (4) an elastic modulus, (5) one or more ratios that include any one of the foregoing, or any combination of the foregoing. The processor may optionally compare (1), (2), (3), (4), or any combination thereof to one or more comparative quantities, e.g., a control sample. The processor may perform the preceding using a CT scan.

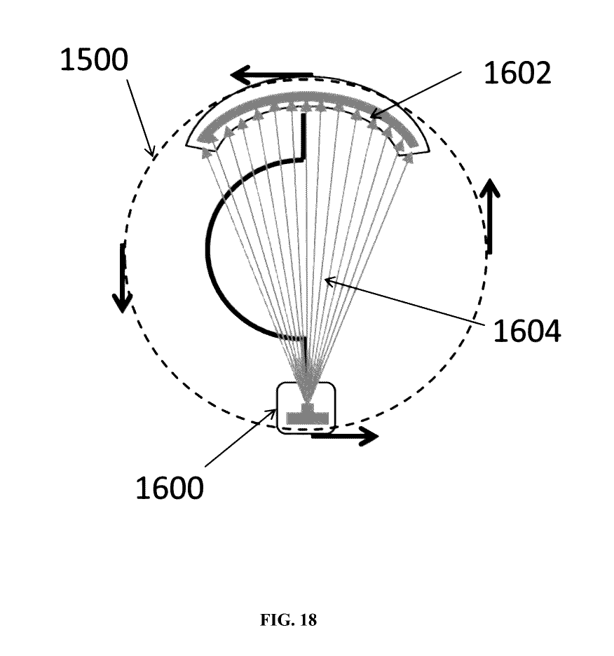

FIG. 18 provides an alternative depiction of the components of a scanner component; the dashed circle shows the trajectory of the x-ray source and detector rotating withing toroid 1500 so as to encircle a sample, with the rotating being in the direction of the counterclockwise arrows. As shown, x-ray source 1600 and detector 1602 rotate in a counterclockwise direction about a sample, with x-rays 1604 directed from source 1600 toward detector 1602. It should be understood that toroid 1500 may be fully circular (e.g., donut-shaped), but may also be C-shaped or otherwise less than a full circle in configuration.

The disclosed systems may include a CT scanner. Suitable CT scanner devices are known to those of ordinary skill in the art.

The processor may be configured to assess a degree of osteoarthritis, osteoporosis, or both, in the bone sample. This may be based on the value of a characteristic (e.g., any one or more of items (1)-(5) listed above) of the sample, on a change in a characteristic of the sample, or even a difference between the characteristic of the sample and a complementary characteristic of another sample or of the same sample taken at a different location in the sample or at a different point in time.

The processor may, in some instances, be configured to determine, for the bone sample, one or more of (1) a volume ratio of trabecular bone rod to trabecular bone plate, (2) a ratio of trabecular bone rod volume to total bone volume.

### ILLUSTRATIVE EMBODIMENTS

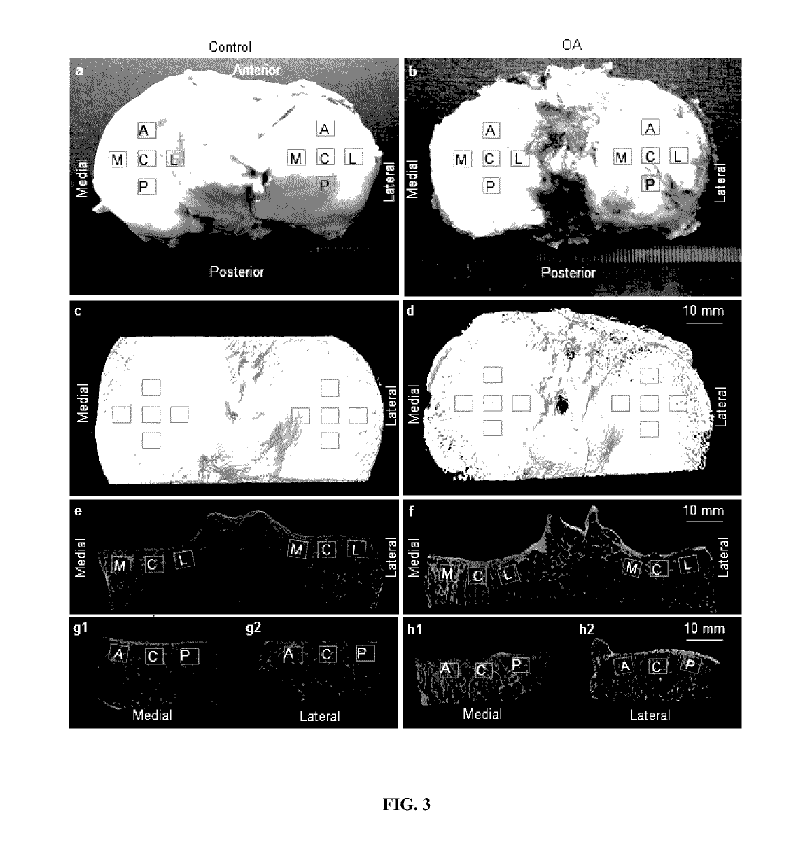

FIG. 3 provides data from tibial plateaus from normal and OA-affected human subjects. Consistent with previous studies, OA is more severe on the medial side with apparent loss of articular cartilage and subchondral sclerosis.

FIG. 3 provides macroscopic and radiographic view of an exemplary tibial plateau from non-OA donors (n=1, left column) and severe OA patients (n=78, right column). 5-mm cubic subregions, marked by squares, from medial and lateral sides of each sample were selected and analyzed. Subregion labels A, M, C, L, and P represent anterior, medial, central, lateral and posterior, respectively.

Corresponding Micro-CT top views (c and d), coronal section through medial, central and lateral subregions (e and f), and sagittal sections through anterior, central, and posterior subregions (g1, g2, h1 and h2) are also provided. Intact full-thickness cartilage covering the entire subchondral bone surface was observed on the normal non-OA knee, while severe cartilage damage was observed on the medial side of OA-affected knee. On the lateral side of OA-affected tibial plateau, osteophytes, an indication of severe OA, were observed, but cartilage appeared mostly intact.

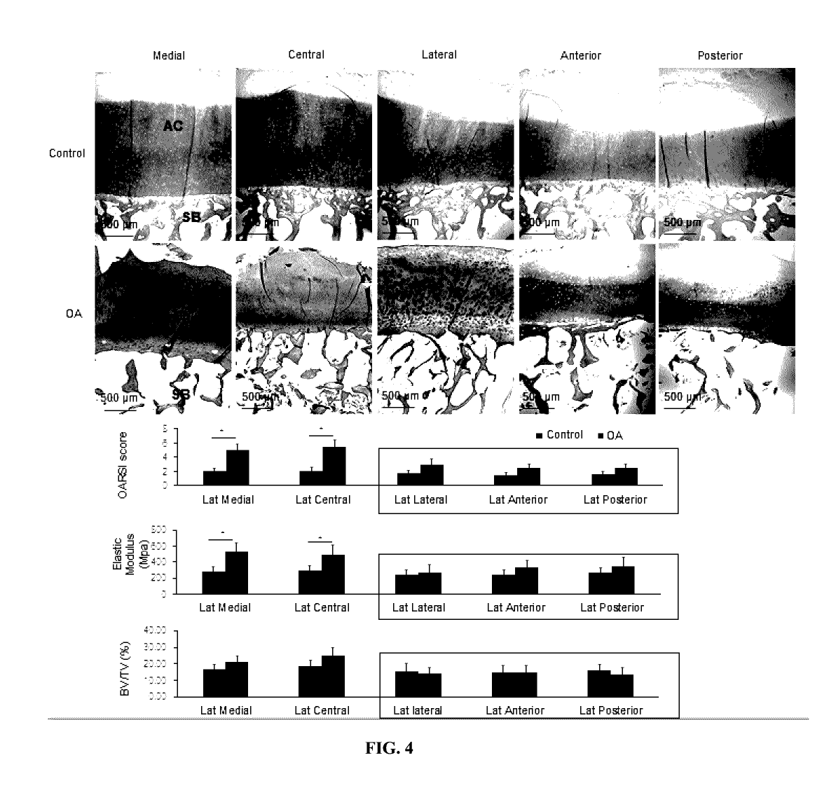

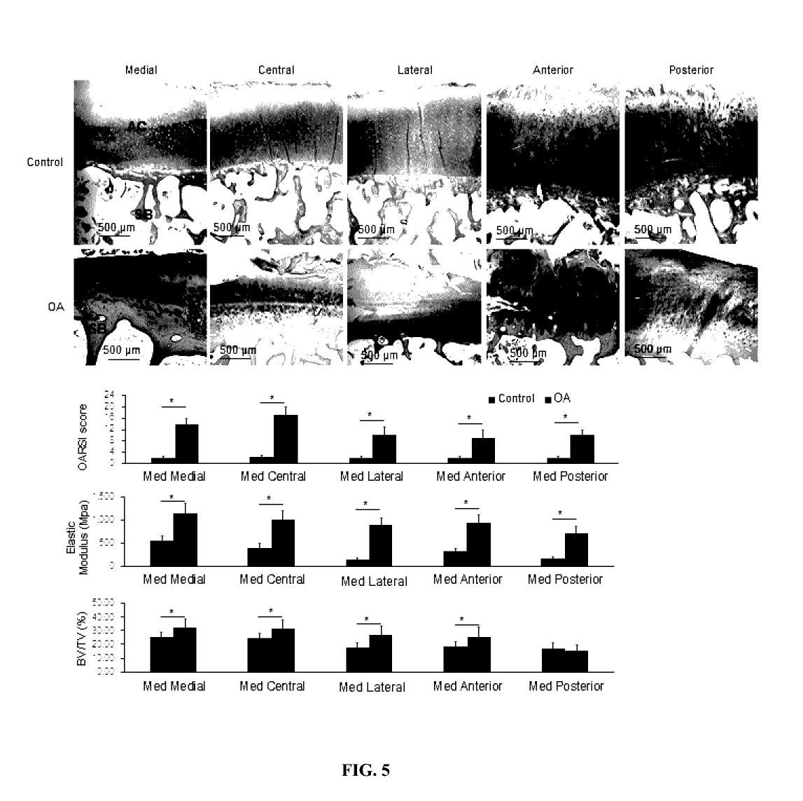

Results (FIGS. 4 and 5) from BV/TV, SMI and elastic modulus calculations of subchondral bone underlying these intact cartilage subregions indicate no significant difference when compared to healthy non-OA controls.

Histological images and finite element analysis of lateral and medial sides of tibial plateau from non-OA and OA-affected knees (FIGS. 4 and 5). Sections were stained with Safranin O and Fast Green to differentiate articular cartilage (AC, red) from subchondral bone (SB, green).

On the lateral side (FIG. 4), compared to normal non-OA sections, cartilage on OA-affected sections is mostly intact and maintains full thickness in the lateral, anterior and posterior subregions, but appears damaged in the medial and central subregions. OARSI score and finite element analysis show consistent results that there is significant difference in the severity of OA and elastic modulus between the lateral sides of normal knee and OA-affected knee only in the medial and central subregions. Notably, no significant change is detected in bone volume fraction (BV/TV) in any of the five subregions.

On the more OA-affected medial side (FIG. 5), cartilage is severely damaged compared to normal knee. Significant difference in OARSI score of cartilage, elastic modulus and bone volume fraction of subchondral bone between normal knee and OA-affected knee was detected in all five subregions (* indicate p<0.05 between non-OA and OA group).

Almost all of the traditional bone parameters (BV/TV, SMI and elastic modulus) showed the subchondral bone underlying severely damaged cartilage to be significantly different from non-OA controls. The high correlation between histological changes in cartilage and traditional measures of the subchondral bone confirmed that osteoarthritic changes in cartilage accompany changes in subchondral bone.

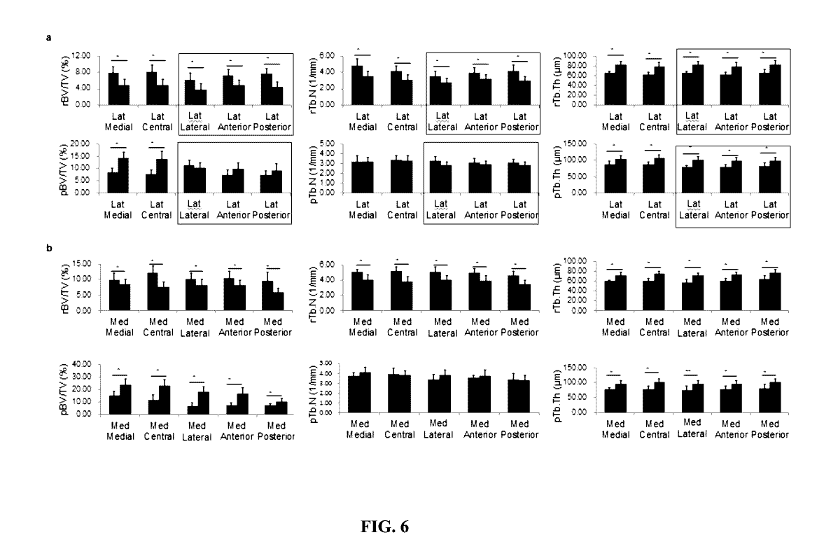

FIG. 6 provides ITS analysis (ITS) of lateral and medial side subchondral bone in non-OA and OA-affected knees from FIGS. 4 and 5. On the lateral side (FIG. 6a) where no differences were observed using FEA and histology, significant differences in rod bone volume density (rBV/TV), rod trabecula number (rTb.N), rod trabecula thickness (rTb.Th) and plate trabecula thickness (pTb.Th) were detected using ITS between normal knee and OA knee in the lateral, anterior and posterior subregions. In particular, there was observed reduction in the number of trabecula rods, and simultaneous thickening of trabecular rods and plates.

On the medial side (FIG. 6b), significant differences in rod and plate volume density, rod and plate trabecula thickness, and rod number were detected. Also observed was a reduction of rod number, and simultaneous thickening of rods and plates in all 5 subregions (* indicate p<0.05 between non-OA and OA group).

Significant changes in several ITS parameters such as rod bone volume fraction (rBV/TV), rod trabecular number (rTb.N) and thickness (rTb.Th), and plate trabecular thickness (pTb.Th) were detected in subchondral bone underneath intact cartilage, where no change was detected by traditional subchondral bone property measurements. In these regions, significant reduction in rBV/TV compared to non-OA control was observed while plate bone volume fraction (pBV/TV) was not different. Changes in rBV/TV and pBV/TV may result from changes either in the number of trabeculae or in the thickness of trabeculae. The data suggest that the reduction in rBV/TV was due to a significant loss in the number of trabecular rods. On the other hand, rTb.th and pTb.th both increased significantly. Further analyses of individual trabecular plates and rods orientation suggested that loss of trabecular rods was uniform along all directions while loss of plates was mainly in the transverse direction parallel to the joint surface (data not shown). The observation that there is no change in pBV/TV can thus be explained by a counter-balance between a reduction in the number of transverse plates and an increase in thickness of the remaining plates.

In subchondral bone under severely damaged cartilage, we observed expected increases in pBV/TV and BV/TV which explain bone sclerosis. This increase in bone density is due to thickening of both trabecular plates and rods. Interestingly, significant rod loss was still detected and was persistent in all subregions. Orientational analyses in these severe OA regions suggested similar loss of transverse plates but a dramatic increase in the number of longitudinal plates (data not shown). These results suggest that trabecular rod loss may be an early and consistent characteristic in the pathogenesis of OA.

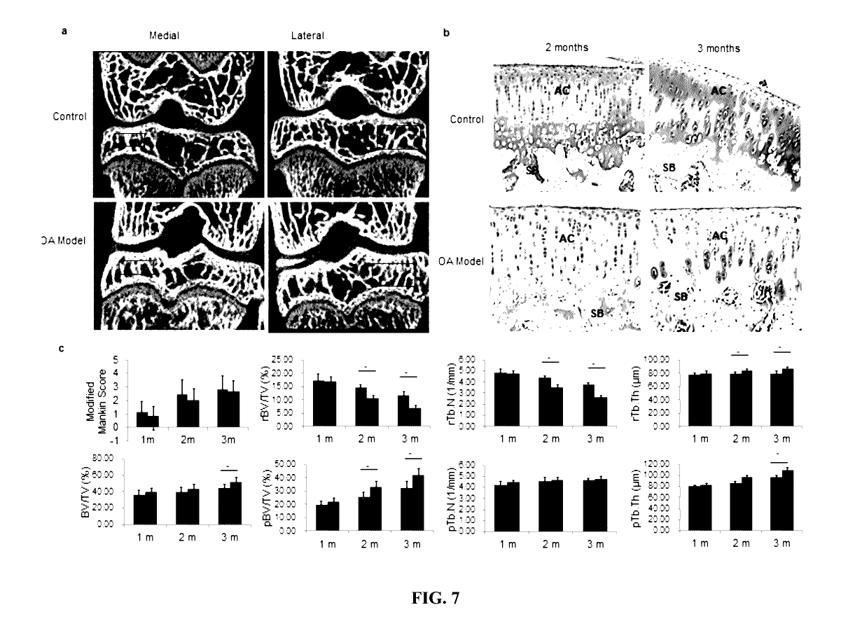

FIG. 7 provides radiographic images, histology, and ITS analysis of tibial plateaus from a guinea pig study of Dunkin Hartley animals and control animals; Dunkin-Hartley guinea pigs are known as a robust model for OA. Six animals were used for each category shown in the test matrix.

Though the data from human patients suggested that microstructure changes, particularly in rods, may initiate prior to cartilage degradation, all samples were from terminal knee OA patients, therefore a timeline was difficult to establish in all cases. Therefore, to elucidate the temporal relationship between cartilage degeneration and subchondral bone remodeling, the hypothesis was tested in a guinea pig OA model. In this study, Dunkin-Hartley guinea pigs, a strain that spontaneously develop OA around the age of 4 months, were compared to controls that do not develop OA spontaneously. At month 2, no articular cartilage degeneration was observed according to Mankin scores and no change in BV/TV in the subchondral bone was detected compared to control (FIG. 4). However, reduction in rBV/TV, accompanied by increase in pBV/TV, was already apparent at this time point. Mechanisms of these microstructural changes were similar to those observed in the human model: trabecular rods decreased in number and increased in thickness at month 2 before significant OA changes occur in the cartilage. These changes became more apparent in month 3 where increase in the overall BV/TV was observed. The results demonstrate that OA development in guinea pig is similar to that in human model in terms of persistent trabecular rod loss. The results also show that subchondral bone changes precede cartilage degradation in OA pathogenesis in a guinea pig model.

Tibial plateau sections were stained with Toluidine Blue to differentiate articular cartilage (AC, purple) from subchondral bone (SB). Histological analysis (FIG. 7a) show intact cartilage at month 2 in guinea pigs that develop OA spontaneously. Degradation in cartilage was observed at month 3 compared to normal guinea pig tibial plateau (FIG. 7b). ITS analysis (FIG. 7c) was performed on selected subchondral bone regions, marked by the rectangles in FIG. 7a. Significant differences were detected between non-OA and OA-affected sample as early as month 2 in rod and plate bone volume density, rod number, and rod thickness. Observed were early rod reduction and rod thickening prior to the development of OA at month 3 (* indicate p<0.05 between non-OA and OA group).

As shown in FIG. 7c, there was a significant difference between control and OA animals in BV/TV at 2 months and in BV/TV and pBV/TV at 3 months. Also as seen, both BV/TV and pBV/TV grew over time for both control and OA animals.

FIG. 7c also provides rBV/TV results for control and OA animals at 1, 2, and 3, months. As shown, there was a significant difference between control and OA animals in both characteristics at 2 and at 3 months. rBV/TV decreased with time for all animals.

FIG. 7c additionally shows pTb.N (plate trabecular number) and rTB.N (rod trabecular number) results for control and OA animals at 1, 2, and 3 months. The difference in rTb.N between control and OA animals was significant at 2 and 3 months. As seen, rTb/N decreased with time for all animals. pTb.N remained relatively constant with time.

Also shown in FIG. 7c are pTb.Th (model-independent plate trabecular thickness) and rTb.Th (model-independent rod thickness) results for control and OA animals at 1, 2, and 3 months. The difference in pTb.Th between control and OA animals was significant at 2 and 3 months, and the difference in rTb.Th between control and OA animals was significant at 3 months. pTb.Th increased with time for all animals, but rTb.Th remained relatively constant over time.

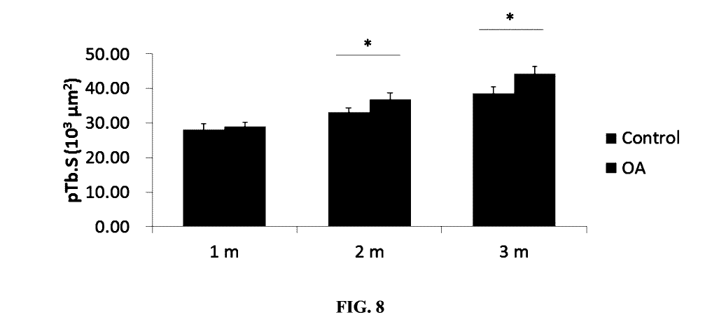

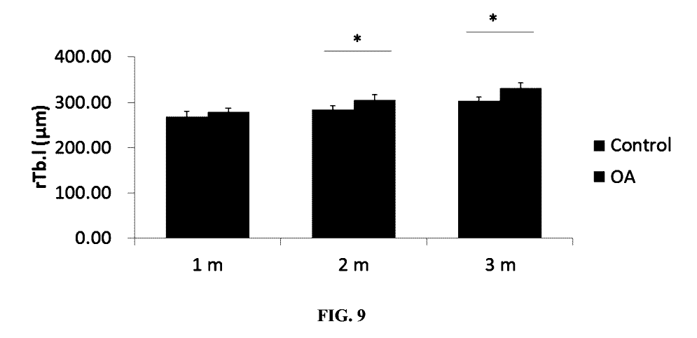

FIGS. 8 and 9 provide pTb.S (plate trabecular spacing) (FIG. 8) and rTb.l (mean trabecular rod length) (FIG. 9) results for control and OA animals at 1, 2, and 3 months. The difference in pTb.S between control and OA animals was significant at 2 and 3 months, and the difference in rTb.l between control and OA animals was significant at 2 and 3 months. pTb.S increased with time, and rTb.l showed some increase over time.

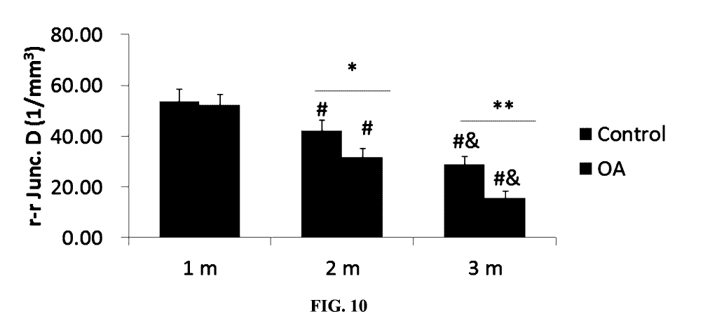

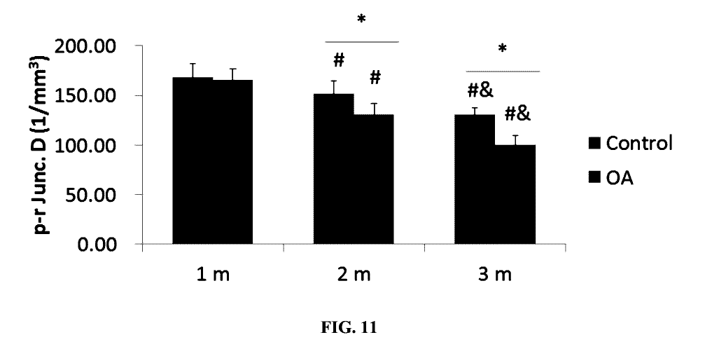

FIGS. 10 and 11 provide r-r Junc.D (rod-rod junction density) (FIG. 10) and p-r Junc.D (plate-rod junction density) (FIG. 11) results for control and OA animals at 1, 2, and 3 months. The difference in r-r Junc.D between control and OA animals was significant at 2 and 3 months, and the difference in p-r Junc.D between control and OA animals was significant at 2 and 3 months. The r-r Junc.D was seen to decrease with time, as was p-R Junc.D.

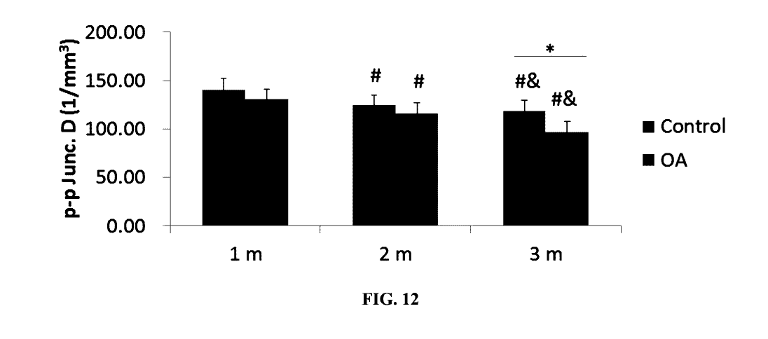

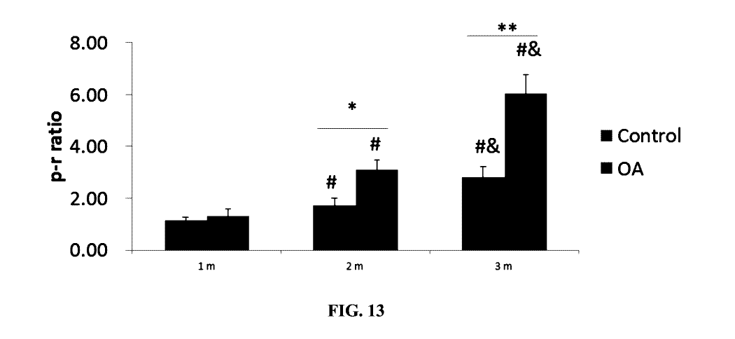

FIGS. 12 and 13 provide p-p Junc.D (plate-plate junction density) (FIG. 12) and p-r ratio results (FIG. 13) for control and OA animals at 1, 2, and 3 months. The difference in p-p Junc.D between control and OA animals was significant at 3 months, and the difference in p-r ratio between control and OA animals was significant at 2 and 3 months. The p-p Junc.D was seen to decrease over time, and the p-r ratio was seen to increase over time.

The foregoing animal data is consistent with the hypothesis that bone remodeling at the rod/plate level takes place with the development of OA. Without being bound to any particular theory, an early sign of abnormal subchondral trabecular bone remodeling in OA begins with a loss of trabecular rods before any significant changes in BV/TV. These lead to adaptive bone responses by dramatically thickening of remaining trabecular plates and rods, resulting in subchondral bone sclerosis and stiffening. The resulting subchondral trabecular bone can be characterized as sparsely spaced thick trabecular plates in the axial direction, providing an uneven and ridged subchondral bone bed for overlaying cartilage. A relative decrease in rod presence may also be seen. These trabecular plate and rod changes in OA provide morphological and biomechanical clues of abnormal subchondral bone remodeling in the etiology of osteoarthritis.

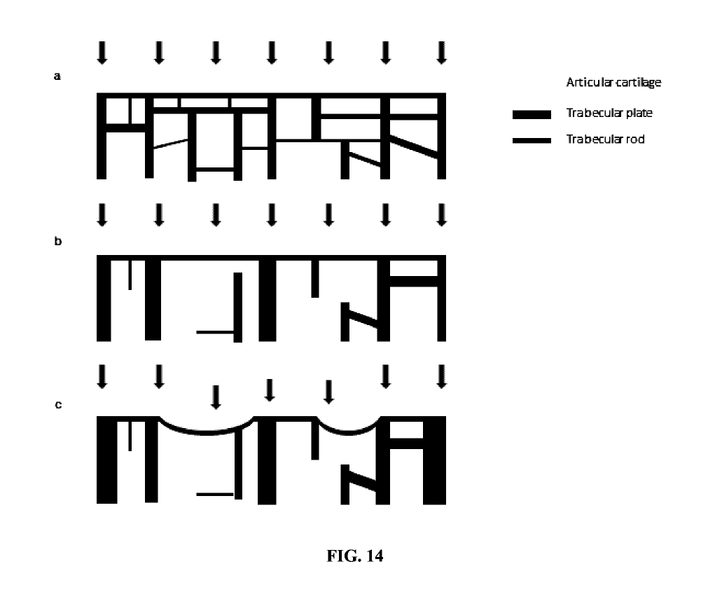

FIG. 14 provides a non-limiting schematic for OA pathogenesis. Healthy subchondral trabecular bone (a) consists of both trabecular plates and rods that provide an even and adequate load-supporting bed for articular cartilage. Rods are distributed preferentially along the transverse direction while plates are highly aligned in both the transverse and longitudinal direction. Joint loads are diverted from the axial direction to the transverse direction, dissipated through the transversely aligned plates and rods. At early stage OA (b), abnormal bone resorption leads to reduction in the number of trabecular rods, and eventual loss of transverse plates due to loading. As the disease progress to a later stage (c), remaining trabeculae increase thickness to keep up with mechanical demand. These changes disrupts the continuous support bed, creating an unfavorable mechanical environment for articular cartilage, and results in cartilage damage.

Exemplary Operation

As described, the present disclosure provides, inter alia, systems for computed tomography of an extremity of a patient, the apparatus comprising: a digital radiation detector and a radiation source. The radiation path from the radiation source to the detector is suitably long enough to allow adequate radiation exposure of the first extremity while maintain the first extremity roughly in the center of the apparatus for an image capture by the detector. A system may be relatively small so as to allow a patient to comfortably position the second extremity to be outside the apparatus during the image capture and also to permit mobility of the system. The disclosed systems provide higher resolution images compared with conventional CT scanners of similar dimensions, thus making it possible for a more detailed trabecular bone microstructural analysis, as well as more reliable information for clinical diagnosis. The systems may be portable but also maintain stability and ensure low noise during a scan.

In an example operation, before a scan the scanner is moved to the desired location and may be fixed in place. A cast designed for holding and fixing the knee/ankle/elbow during a scan may be used to reduce relative movement of the patient; the cast may be custom or may be adjustable to accommodate different patients. A patient may sit on an adjustable chair specifically designed for patients with different sizes and puts the body part to be scanned in the corresponding cast. The position of the chair and the cast are then adjusted to place a patient's leg/ankle/elbow in the center of the scanning area while ensuring the patient's comfort. A CT scanner may perform a quick preliminary or “scout” scan to locate the region of interest; the preliminary scan may take only a few seconds to perform. Then the main scan starts with a scanning protocol designed for the site(s) of interest, e.g., knee, elbow, and the like.

A standard scan region may be, e.g., 10 mm and a standard scan time may be 2 minutes. During the scan, the pair of radiation source and detector may rotate and encircle a patient's knee/elbow/ankle, etc. The radiation source and detector are actively emitting and receiving x-rays during this process. A set of 2D stack images are automatically reconstructed after the scan for further imaging analysis and clinical diagnosis.

### SUMMARY

Morphological and biomechanical integrity of subchondral bone is essential, because it serves as an energy and shock absorber that transmit joint load. In the healthy joint, subchondral plates and rods, two components that make up trabecular bone, form a unique orientational distribution: rod trabeculae are aligned along the transverse direction (parallel to joint surface), while plate trabeculae are aligned along both the transverse and longitudinal direction. This preferential arrangement shunts the mechanical forces from the longitudinal direction and dissipates these loads through the transverse plates and rods. Disruption in the microstructural meshwork of plates and rods may present unfavorable results mechanically to the joint.

As described herein, one may use ITS to analyze microstructural changes in the subchondral bone in normal and OA-affected knee, and discovered a significant and persistent loss of rod-like trabeculae at the early stage of OA before articular cartilage degeneration could be detected. Without being bound to any particular theory, this observation may serve as a marker in the initial stages of OA. Besides trabecular rod loss, thickening in both trabecular plates and rods was also observed. The interplay between bone loss in the form of rod reduction and bone gain in terms of trabeculae thickening offers an explanation for the paradoxical question of how abnormal resorption patterns can lead to eventual bone sclerosis. This represents a critical improvement over existing models, as this model provides a mechanistic explanation for OA progress, which explanation also provides pathways for treatment options.

One may postulate a mechanistic model for OA pathogenesis (FIG. 14). At the early stage of OA, uncoupled bone resorption attacks trabecular rods and transverse trabecular plates. Trabecular rods are more vulnerable as they have higher surface-to-volume ratio and are relatively thinner than trabecular plates. The loss of transverse trabecular plates may result from unloading in various pathological conditions. Decrease in the number of load supporting trabeculae results in higher load and mechanical demand distributed among the remaining trabeculae, and leads to trabeculae thickening in the short term. These structural changes disrupts the original supporting bed in the long term and presents an unfavorable mechanical condition such as local stress concentrations in the cartilage. These mechanisms provide a basis for a slow but progressive development of OA in the articular cartilage.

The disclosed data show that analyzing the microstructure of subchondral bone using ITS offers new and unexpected insight into the pathogenesis of OA. As discussed, even in patients with advanced OA, it was observed that in addition to regions with severely damaged cartilage, there were also regions (particularly the posterior, anterior, and lateral portions of the lateral plateaus) where articular cartilage was intact according to Osteoarthritis Research Society International (OARSI) scores. Measures of BV/TV, SMI, and elastic modulus of subchondral bone did not differ between these intact regions and healthy controls. However, ITS analyses detected significantly and unexpectedly lower rod bone volume fraction (rBV/TV) in the regions under intact cartilage, primarily due to lower trabecular rod number (rTb.N).

Beneath the damaged cartilage, OARSI scores, BV/TV, SMI, and elastic modulus did differ significantly from normal controls. However, rBV/TV was significantly lower due to lower rod trabecular number (rTb.N), comparable to that detected underneath intact cartilage. These observations suggest loss of trabecular rods may precede cartilage deterioration in OA. Though one might not consider the regions with intact cartilage from OA affected knees as “normal”, results from spontaneous OA guinea pig models also indicate trabecular rod loss precedes cartilage damage. Based on these findings, one may postulate that abnormal uncoupled bone resorption causes trabecular rod loss that precedes cartilage damage. As trabecular rods are lost in subchondral bone, increased joint load sharing likely leads to a thickening of remaining trabecular plates. As subchondral bone stiffness increases, there is increasing tissue stress in the overlying articular cartilage, leading to damage accumulation and progression to OA. This explanation—and the associated methods of diagnosis and treatment—represents a quantum improvement over existing techniques, which techniques—as discussed—may not identify OA until OA has progressed in a significant way. Indeed, the disclosed technology (owing to the key insight that bone may remodel below intact cartilage) allows for recognition and treatment of OA before a patient may experience physical symptoms.

These disclosed findings and discoveries from present improved therapeutic options for the treatment of OA. As one example, administration of anti-resorptive drugs at the onset of OA when signs of microstructural changes are detected—but also when a patient is asymptomatic or has little to no symptoms—may alleviate the loss of rod trabeculae and prevent subsequent bone sclerosis and cartilage degradation. These results provide a logical explanation of how early bone loss can lead to later bone sclerosis and suggest a new model for OA pathogenesis.

The disclosed systems also provide several improvements over existing alternatives. First, the disclosed systems provide for comparatively low radiation exposure studies that still yield sufficiently detailed information to allow medical personnel to assess and/or treat OA. Second, the disclosed systems, by virtue of their size and portability, allow for diagnostic procedures to be carried out on-site (e.g., in a patient's room or in the field) without forcing a patient to travel to a radiological diagnostic center. Further, the disclosed systems are sized so as to allow for studies of joints (e.g., knees, elbows, and other joints) that are affected by OA.

What is claimed:

1. A diagnostic system, comprising:
a toroid tomography scanner, the toroid having a central axis and an inner diameter about the central axis,
the inner diameter of the toroid being so dimensioned as to permit entry of a human limb such that a joint of the limb is disposed within the toroid,
the toroid comprising an x-ray source and an x-ray detector, the source and detector configured to be in x-ray communication with one another during operation,
the system being configured such that one or more of the x-ray source, the x-ray detector, and the toroid are rotatable about the limb disposed within the toroid.

2. The system of claim 1, wherein the system is configured to operate as a high resolution peripheral quantitative computed tomography system.

3. The system of claim 1, wherein the system is configured to output at a resolution of from about 100 to about 40 microns per voxel.

4. The system of claim 1, wherein the toroid is mounted on a portable chassis.

5. The system of claim 1, wherein the system is configured to provide x-rays of two or more different energies to the joint.

6. The system of claim 1, wherein the toroid has a thickness of from about 250 to about 350 mm.

7. The system of claim 1, wherein the inner diameter of the toroid is in the range of from about 250 to about 350 mm.

8. The system of claim 1, wherein the system further comprises a processor operatively connected to the toroid, the processor being configured to determine, based on structural information of a bone of the joint of a subject, one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing.

9. A diagnostic system, comprising:
a processor configured to determine, structural information of a bone sample, one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing.

10. The system of claim 9, further comprising an imaging device capable of providing structural information for the processor.

11. The system of claim 10, wherein the imaging device is characterized as a high-resolution peripheral quantitative CT scanning device.

12. The system of claim 11, wherein the CT scanning device is operated so as to provide x-rays of two or more different energies to the bone sample.

13. A method, comprising:
(a) determining, for a bone sample, one or more of (1) a volume fraction of trabecular bone plates to total bone volume, of trabecular bone rods to total bone volume, or both, (2) a cross-sectional dimension of trabecular bone plate, of trabecular bone rod, or both, (3) a number density of trabecular bone plates, of trabecular bone rods, or both, (4) an elastic modulus, and (5) one or more ratios that include any one or more of the foregoing;
(b) comparing (1), (2), (3), (4), (5) or any combination thereof to one or more comparative quantities; and
(c) estimating a degree of osteoarthritis, osteoporosis, or both, of the bone sample.

14. The method of claim 13, wherein the determining is performed with a high-resolution peripheral quantitative CT scanning device.

15. The method of claim 13, wherein the determining is effected by providing x-rays of two or more different energies to the bone sample.

16. The method of claim 13, further comprising determining (1), (2), (3), (4), (5), or any combination thereof for two or more locations within the bone sample.

17. The method of claim 13, further comprising selecting a treatment regimen based at least in part on the estimate of osteoarthritis, osteoporosis, or both, in the bone sample.

18. The method of claim 13, wherein the method is performed on a subject who is asymptomatic for osteoarthritis.

19. A method, comprising:
providing a CT scanning device operatively connected to an osteoarthritis estimation component that comprises a processor;
operating the CT scanning device so as to obtain bone structural information from a bone sample;
outputting the bone structural information to the osteoarthritis estimation component; and
executing instructions on the processor of the intracranial pressure estimation component, the instructions including
(a) receiving the bone structural information, and
(b) generating from that information one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, and
estimating a degree of osteoarthritis, osteoporosis, or both, in the bone sample from the one or more foregoing quantities (1)-(5).

20. A method of treating osteoarthritis, comprising:
administering a therapeutically effective dose of an osteoarthritis treatment to a patient who is asymptomatic for osteoarthritis and exhibits one or more of (1) a volume fraction of trabecular bone plate to total bone volume, of trabecular bone rods to total bone volume, or both, (2) a cross-sectional dimension of trabecular bone plates, of trabecular bone rods, or both, (3) a number density of trabecular bone plates, of trabecular bone rods, or both, (4) an elastic modulus, (5) one or more ratios that include any one or more of the foregoing, wherein the foregoing characteristic of the patient differs from one or more comparative quantities.
